(19) United States
(12) Patent Application Publication
(10) Pub. No.: US 2012/0141611 A1
(43) Pub. Date: Jun. 7, 2012

(54) METHODS AND COMPOSITIONS USING ERGOTHIONEINE TO TREAT A VARIETY OF HEALTH RELATED FACTORS

(75) Inventors: Bernard Martin Landes, Laguna Niguel, CA (US); Michael A. Finamore, Roslyn Harbor, NY (US)

(73) Assignee: Oxis International Inc.

(21) Appl. No.: 13/311,528

(22) Filed: Dec. 5, 2011

Related U.S. Application Data
(60) Provisional application No. 61/419,872, filed on Dec. 5, 2010.

(51) Int. Cl. A61K 36/889; A61K 31/7008; A61K 36/9066; A61K 36/67; A61P 29/00; A61K 38/39; A61K 31/726; A61K 36/74; A61K 36/76; A61K 31/4172; A61K 31/714; A61K 31/728

(52) U.S. Cl. 424/727; 514/52; 514/62; 424/756; 424/734; 514/54; 514/17.2; 424/725; 424/769; 514/392

(57) ABSTRACT

In one embodiment, a composition containing ergothioneine and/or synthesized l-ergothioneine and one or more compounds selected from the group consisting of vitamin B-12, glucosamine sulfate, calcium ascorbate, turmeric extract, black pepper extract, hyaluronic acid, collagen, glycosaminoglycans, cat's claw extract, acai berry; and white willow bark extract is provided to a mammal to improve a variety of health related factors, including but not limited to brain health, joint health, eye health, mitondchorial optimization/improvement and reduction of inflammation and pain in a mammal.

### CROSS REFERENCE To RELATED APPLICATIONS

This application claims benefit of and priority to U.S. Provisional Application No. 61/419,872, entitled, “METHODS AND COMPOSITIONS USING ERGOTHIONEINE TO TREAT A VARIETY OF HEALTH RELATED FACTORS,” filed Dec. 5, 2010, the entirety of which is hereby expressly incorporated herein by reference.

### BACKGROUND

1. Technical Field

The present disclosure relates generally to the use of ergothioneine and/or its derivatives, compositions containing ergothioneine and related compounds for a variety of health related compounds. More particularly, the present disclosure is related to the use of, ergothioneine, l-ergothioneine and 99.9% pure synthesized l-ergothioneine and compositions containing a combination of one or more of these compounds with any combination selected from the group consisting of natural compounds including, but not limited to, vitamin B-12, glucosamine sulfate, calcium ascorbate, turmeric extract, black pepper extract, hyaluronic acid, collagen, glycosaminoglycans, cat's claw extract, acai berry, and white willow bark extract to improve a variety of health related factors, including, but not limited to, brain health, joint health, eye health, mitochondrial optimization/improvement and to reduce inflammation and pain.

2. Description of Related Art

Aspirin is one of the oldest known pain relievers. However, it is not effective for severe pain and more importantly, it poses a danger to one's stomach. The salicylic acid in aspirin can cause potentially deadly stomach bleeding, ringing in the ears, impaired kidney function, and liver damage. Acetaminophen is often considered a more modern form of aspirin. Acetaminophen products raise one's pain threshold, so more pain is required before one's brain recognizes it. Acetaminophen also has a long list of dangerous side effects, ranging from elevated blood pressure to allergic reactions, including the most dangerous, liver damage.

Creams and ointments for joint pain, which have cap saicin as a main ingredient, lead to a warm sensation after application, but have no effect on cartilage, ligaments, or any other joint compound. Capsaicin interferes with the transmission of pain along the nerves, so while the pain is not registering with one's brain, the overall health and functionality of the joint has not improved.

Prescription pain relievers are notorious for their side effects, such as increased risk of heart attack and stroke, myoclonus, hallucinations, increased risk of osteoporosis, cataracts, glaucoma and high blood pressure. Patients whose symptoms still persist increasingly resort to joint replacement surgery, but joint implants wear out over time and need to be replaced, leading to more surgery. Such surgeries also subject the patients to the potential of severe post-operative pain, infection, and blood clots. Patients also report their new joint is stiff and clumsy, or that their replacement is loose or dislocated.

L-Ergothioneine is a unique, naturally occurring antioxidant that is abundant in most plants and animals. Ergothioneine is synthesized by bacteria and fungi in the soil and cannot be synthesized by humans or plants. Therefore, it is available only from dietary sources, which provide miniscule amounts of l-ergothioneine in unpredictable amounts. Synthetic l-ergothioneine can be utilized to provide consistent levels of ergothioneine via a wide variety of delivery forms, including dietary supplements, functional foods and beverages and even topical delivery systems. The avidity by which dietary ergothioneine is assimilated by tissues, the specific and significant effects it has on cellular processes, and the degree to which it is conserved by cells suggest an important physiological role for this molecule. It has been shown that ergothioneine plays a unique dual role in both energy regulation and in protecting cells from oxidative damage.

High concentrations of ergothioneine are found in a number of organ systems including the liver, kidney, eye, seminal fluid, and erythrocytes. The role and biological significance of ergothioneine is the subject of an increasing number of scientific studies that, over time, will lead to a more complete understanding of the full range of its functions and benefits.

Technically classified as an amino acid, ergothioneine has extraordinary properties that set it apart from any other compound found in nature. It is the most powerful antioxidant ever discovered, more potent than glutahione and vitamin E, long considered the strongest antioxidants known to science. It helps other antioxidants in the body to regenerate and work longer in the body. It works as a metabolic regulator, rejuvenating tissues at the cellular level and helping to optimize energy protection. Lastly, it has its own gene (SLC22A4) that comes to life in times of elevated oxidative stress to create a genetic transporter (OCTN1) specifically designed to carry ergothioneine to the location in the body where its functionality can be most beneficial.

When oxidative stress and inflammation are present in the body, the SLC22A4 gene creates a protein that seeks out and attaches itself to ergothioneine and then transports the ergothioneine to the site of the oxidative stress and inflammation. Ergothioneine is the only substance that functions this way with its own dedicated genetic transporter, suggesting that nature intends it to be the body's primary defense against oxidative stress and inflammation.

Ergothioneine is a highly protective, nontoxic, naturally occurring antioxidant that is not easily auto-oxidizable in aqueous solutions. Ergothioneine is unique among antioxidants in that it chelates heavy metal, while protecting cells (principally erythrocytes) from ROS damage.

Ergothioneine is a sulfur-containing amino acid derivative of histidine, which was first isolated from an ergot fungal infection (Claviceps purpurea) of rye grain in the early 1900s. High levels are found in certain mushrooms such as Porcino (Boletus edulis [528 mg/kg]) and oyster mushrooms (119 mg/kg), and moderate levels are found in foods such as black beans (13.49 mg/kg), kidney beans (4.52 mg/kg), oat bran (4.41 mg/kg) and garlic (3.11 mg/kg). It is present in plants, animals and humans; however, because only certain bacteria and fungi can synthesize it, it must be obtained through the soil or diet. Because of this, some researchers (Paul, B. D. and S. H. Snyder. “The unusual amino acid L-ergothioneine is a physiologic cytoprotectant.” Cell Death Differ. 2009.) have suggested that it may meet the definition of a vitamin, although no syndrome has thus far been associated with its deficiency.

Ergothioneine is quite stable in the body with a long half-life of approximately thirty days. Its principle metabolite is hercynine. It is not able to pass through the plasma membrane of cells on its own—it requires a special transporter protein. Ergothioneine is the key substrate for the transporter protein OCTN1. Cells that are depleted of the transporter protein are more susceptible to oxidative stress, resulting in DNA damage, and protein and lipid oxidation.

If ergothioneine is a physiological antioxidant, depletion of its transporter should result in the decreased ability of cells to cope with oxidative stress. The augmented cytotoxicity associated with its transporter, OCTN1, depletion indicates that basal levels of ergothioneine provide physiologic cytoprotection. Paul and Snyder's study provides substantial evidence that ergothioneine is a physiologic antioxidant cytoprotectant. OCTN1 maintains ergothioneine tissue levels. Endogenous levels of ergothioneine vary, attaining millimolar concentrations in tissues that are typically exposed to marked oxidative stress such as blood cells, the lens of the eye, the liver and bone marrow. Appreciation of ergothioneine as a physiologic antioxidant augments an already substantial cohort of such agents. Presumably, a multiplicity of diverse antioxidants helps the cell to cope with a wide range of stresses. Ergothioneine may afford a more stable mode of cytoprotection. Its stability may help mitochondria cope with otherwise overwhelming stresses encountered even during relatively physiologic metabolism. In this sense, ergothioneine probably fits the definition of a vitamin. In summary, ergothioneine is a most unusual amino acid with substantial antioxidant efficacy.

The high density of OCTN1 in mitochondria implies a unique role in protecting this organelle from the reactive oxygen species that accumulate even with normal oxidative metabolism. Ergothioneine also protects the cell from damage induced by reactive nitrogen species and UV radiation. For all these reasons ergothioneine appears to be an important physiologic cytoprotectant, which probably merits designation as a vitamin.

Ergothioneine, whose chemical formula is C9H15N302S, has a molecular weight of 229.31 g/mol. As a white solid, it has a melting point of 260° C., and is composed 46.72% of carbon, 6.45% of hydrogen, 18.11% of nitrogen, and 13.78% of sulfate.

Accordingly, there exists a need for compositions to improve a variety of health related factors, including, but not limited to, brain health, joint health, eye health, mitochondrial optimization/improvement and to reduce inflammation and pain in a mammal. The present disclosures provide these and other benefits.

### SUMMARY

Ergothioneine appears to function as an antioxidant in cells, and can protect against damage by ultraviolet radiation. It concentrates in the liver, where it may protect liver cells from damage, and support its healthy and normal structure and functioning. Ergothioneine has also been shown in animal studies to protect the eyes from toxicity. Ergothioneine is able to chelate divalent metals, and can protect against the formation of free radicals resulting from reactions with these metals.

One aspect of the present disclosure is to provide commercial applications for the isolated natural antioxidant compound as a dietary supplement.

In an embodiment of the present disclosure, methods using ergothioneine and compositions containing a combination of ergothioneine and any combination of natural compounds selected from the group consisting of, but not limited to, vitamin B-12, glucosamine sulfate, calcium ascorbate, turmeric extract, black pepper extract, hyaluronic acid, collagen, glycosaminoglycans, cat's claw extract, acai berry and white willow bark extract to improve a variety of health related factors, including, but not limited to, brain health, joint health, eye health, mitochondrial optimization/improvement and to reduce inflammation and pain in a mammal is provided.

Another aspect of the present disclosure is to provide a solution to joint pain without side effects, such as damage to the stomach or liver or interference with prescription medication, while addressing the underlying cause of joint pain, relieving inflammation at its root cause and providing elements for the body to naturally rebuild damaged joints.

Another aspect of the present disclosure is to provide compositions containing pure ergothioneine as a health supplement.

Another aspect of the present disclosure is to provide compositions containing pure ergothioneine that help protect against free radical damage.

Another aspect of the present disclosure is to provide compositions containing pure ergothioneine that help prevent oxidative damage to cells.

Another aspect of the present disclosure is to provide compositions containing pure ergothioneine that help provide antioxidant protection.

Another use of ergothioneine is in a health supplement, wherein pure ergothioneine supports optimal cellular health and metabolism.

Another aspect of the present disclosure is to provide compositions containing pure ergothioneine that to protect the integrity of lipids and fats in the face of free radical oxidation.

Another aspect of the present disclosure is to provide compositions containing pure ergothioneine that help enhance the integrity of cellular membranes.

Another aspect of the present disclosure is to provide compositions containing pure ergothioneine that help protect against the oxidation of iron-containing proteins like hemoglobin and myoglobin, allowing them to maintain their important oxygen-carrying capability.

Another aspect of the present disclosure is to provide compositions containing pure ergothioneine that help inhibit the enzymatic activity of polyphenol oxidase in various foods, which could help preserve foods from turning brown.

Another aspect of the present disclosure is to provide compositions containing pure ergothioneine to act as a potent free radical scavenger of peroxynitrite and hydroxyl radicals.

Another aspect of the present disclosure is to provide compositions containing pure ergothioneine to provide healthy inflammatory response by modulating key cytokines.

Another aspect of the present disclosure is to provide compositions containing pure ergothioneine that help enhance the effects of the potent antioxidant vitamin C.

Another aspect of the present disclosure is to provide compositions containing pure ergothioneine that help preserve other endogenous antioxidants, including vitamin E, and the antioxidant glutathione.

Another aspect of the present disclosure is to provide compositions containing pure ergothioneine to help protect DNA, including mitochondrial DNA, from the effects of free radical damage.

Another aspect of the present disclosure is to provide compositions containing pure ergothioneine that helps prevent oxidative stress and its consequences, such as DNA damage, protein oxidation and lipid peroxidation.

Another aspect of the present disclosure is to provide compositions containing pure ergothioneine for eye health.

Another aspect of the present disclosure is to provide compositions containing pure ergothioneine for eye health, wherein ergothioneine may confer potent protection to normal eye health and function. Higher levels of ergothioneine in human lens tissue are associated with healthier eyesight and visual function. Animal studies corroborate the protective effect of ergothioneine on eye health. (Moncaster, J. A., et al., “Ergothioneine treatment protects neurons against Nmethyl-D-aspartate excitotoxicity in an in vivo rat retinal model.” Neurosci Lett, 2002. 328(1): p. 55-9.)

Another aspect of the present disclosure is to provide compositions containing pure ergothioneine to support the well-being of the neurons of the eye.

Another aspect of the present disclosure is to provide compositions containing pure ergothioneine for liver health.

Another aspect of the present disclosure is to provide compositions containing pure ergothioneine to promote healthy liver function.

Another use of pure ergothioneine is in a supplement for liver health, wherein ergothioneine can enhance the protective activity of endogenous antioxidant enzymes in liver cells, such as glutathione peroxidase and superoxide dismutase.

Another use of pure ergothioneine is in a supplement for liver health, wherein ergothioneine may promote liver health by enhancing liver detoxification activity.

Another use of pure ergothioneine is in a supplement for skin health.

Another use of pure ergothioneine is in a supplement for skin health, wherein ergothioneine protects skin cells in the face of oxidative damage from UV rays.

Another use of pure ergothioneine is in a supplement for skin health, wherein ergothioneine protects against both gamma and ultraviolet radiation.

Another use of pure ergothioneine is in a supplement for cognitive health.

Another use of pure ergothioneine is in a supplement for cognitive health, wherein ergothioneine enhances cognitive health and function by promoting the health and normal life cycle of neural cells.

In another embodiment of the present disclosure, a composition containing approximately 250 mcg vitamin B-12 (Cyanocobalamin), 500 mg glucosamine sulfate (from Glucosamine 2HCl), 50 mg Calcium Ascorbate (from Ascorbic Acid and Calcium Ascorbate), 50 mg Turmeric Extract (95% curcuminoids) (Curcuma longa) (root), 2.5 mg Black Pepper Extract (95% Piperine) (Piper nigrum) (fruit) 2.5 mg Hyal-Joint™ (60% Hyaluronic Acid, 30% Collagen, 10% Glycosaminoglycans), 250 mcg ERGO (L-Ergothioneine), 150 mg blend of Cat's Claw Extract, Acai Berry extract and white willow bark extract (Salix alba) is provided to a mammal one a day to provide joint health.

In yet another embodiment of the present disclosure, a composition containing any combination of vitamin B-12, Calcium Ascorbate, Turmeric Extract, Black Pepper Extract, L-Ergothioneine Cat's Claw Extract, Acai Berry and white willow bark extract is provided to a mammal one a day to provide brain health, eye health, mitochorial optimization/improvement and pain relief.

These, as well as other components, steps, features, objects, benefits, and advantages, will now become clear from a review of the following detailed description of illustrative embodiments, and the claims.

### DETAILED DESCRIPTION OF ILLUSTRATIVE EMBODIMENTS

Illustrative embodiments are now discussed. Other embodiments may be used in addition or instead. Details that may be apparent or unnecessary may be omitted to save space or for a more effective presentation. Conversely, some embodiments may be practiced without all of the details that are disclosed.

Ergothioneine is the body's genetically directed first line of defense in the war on free radicals, oxidative stress and the resulting inflammation. Ergothioneine “lends” one of its electrons to a free radical, transforming it from a destructive, volatile molecule into a harmless one. Unlike other antioxidants, ergothioneine doesn't get used up quickly. Instead, it continues to seek out inflammation-causing free radicals for an extended time (with a half-life in the body of 30 days, compared to as little as 30 minutes for other antioxidants). This impressive half-life is one of the reasons it is considered nature's most powerful antioxidant. Numerous recent studies have shown that ergothioneine is a powerful scavenger of free radicals. Researchers say it provides “an important cellular defense against oxidative stress” in human diseases [2].

Studies show that in addition in to being a powerful antioxidant in its own right, ergothioneine helps other antioxidants regenerate themselves, extending their useful life as well. A study published in Clinical Nutrition demonstrated that ergothioneine helps recycle and preserve antioxidants that are present in the body, including vitamin E and the master antioxidant glutathione. The study also indicates that ergothioneine can help protect two vital organs, the liver and kidneys, from oxidation [3]. Researchers in the U.S. and France found that ergothioneine also helps “recycle” vitamin C. Writing in the Biochemical Journal, they concluded that ergothioneine and vitamin C work well together to fight free radical damage [4]. By itself, ergothioneine has a unique ability to support the body in its fight against joint pain and inflammation.

Recent research proves that inflammation also plays a major role in atherosclerosis, the hardening and narrowing of the arteries. Atherosclerosis, in turn, can lead to heart attacks, strokes and other forms of cardiovascular disease. Vascular inflammation is usually blamed on a buildup of so-called “bad cholesterol” or low-density lipoprotein (LDL). While that's clearly a leading cause, inflammation can also be triggered by a variety of other factors, including hypertension, diabetes, and various types of infections.

Although compositions disclosed in the present disclosure are formulated to relieve joint pain and help the body rebuild damaged joints, they also fight inflammation throughout the body, including cardiovascular inflammation. In fact, a recent study showed that the ergothioneine in the present disclosure provides protection against the formation of plaque on artery walls. Thus, yet another benefit of the present disclosure is the applicability of key elements of the formulation to better cardiovascular health.

Joints consist of various types of tissue, such as such as muscles, ligaments, cartilage and tendons, all of which help the bones move as they're supposed to. One of the most important joint components is synovial fluid—a thick liquid with the consistency of an egg yolk. Synovial fluid is made up of hyaluronic acid and a carbohydrate made from glucuronic acid and acetylglucosamine, both of which are first cousins of glycosaminoglycans. Synovial fluid is essential for maintaining healthy, pain-free joints. It helps the joint absorb shocks. It also supplies oxygen and nutrients to surrounding cartilage, and carries away carbon dioxide and metabolic waste from cartilage cells, but its primary function is to reduce friction by lubricating the joint. The synovial fluid is contained within a thin membrane, the bursa sac. As joints move, this synovial fluid passes through the membrane into the joint, coating the surface of the joint with a thin layer, filling into micro-cavities and empty spaces. This helps each joint bend and flex with ease, but when something goes wrong, that movement can become difficult.

Most cases of joint pain are caused when this synovial membrane ruptures or leaks, allowing synovial fluid to escape. Parts of the joint that are normally lubricated then rub against each other. If this continues for an extended period of time, the joint can become permanently damaged. The synovial membrane sometimes ruptures because of trauma to the joint, but most cases are caused by inflammation, which slowly degrades the membrane to the point where it tears or become porous. The disclosed formula is designed to help the body replenish lost synovial fluid by providing a new supply of hyaluronic acid and glycosaminoglycans, the two key raw materials needed to produce and maintain it.

Hyaluronic acid helps end osteoarthritis pain. A study conducted at the University of Western Ontario in Canada examined the effects of hyaluronic acid on 537 patients (average age 68) diagnosed with osteoarthritis. After 27 weeks, hyaluronic acid was shown to significantly reduce joint pain while resting and while walking. Researchers deemed hyaluronic acid “highly effective” for these patients [1]. More importantly, the disclosed formula also provides previously unavailable nutritional support to help the body reduce inflammation, causing the synovial membrane to leak.

Inflammation is the body's natural reaction to harmful stimuli. The cause of the inflammation may be oxidative stress, which occurs when the body is overwhelmed by free radicals—rogue molecules that wreak havoc on cells. These molecules are created as a byproduct of oxidation, the process that occurs when oxygen interacts with cells to produce energy. Free radicals are made up of atoms that have either too few or too many electrons in their outer shell.

The vitamin B12 and ergothioneine both help regulate cellular metabolism, rejuvenating tissues at the cellular level. Ergothioneine is the only antioxidant known to work within the mitochondria, the “power plant” within the cell that supplies it with the energy it needs to function. In addition, the mitochondria are involved in cell growth and death and are believed to play a role in the aging process [16]. Researchers in London found that ergothioneine protects human cells and DNA from being damaged or destroyed by free radicals [17].

The end result is oxidative stress that: 1) produces inflammation that compromises the integrity of the synovial membrane; 2) kills some cells outright, while damaging the DNA of others, including the mitochondria; 3) boosts the body's inflammation response, producing pro-inflammatory proteins known as cytokines in the immune system; and 4) activates “adhesion” molecules in white blood cells that cause them to stick to and destroy normal cells in arteries and joints, instead of the infectious microbes and damaged cells they're designed to attack.

The present disclosure addresses the underlying cause of joint inflammation. It contains a proprietary blend of five antioxidants that work together to overcome oxidative stress and the inflammation it produces. Each of these specially selected ingredients attacks the problem and supports optimal joint function in a different way.

Another ingredient in the present disclosure, turmeric, is a tropical shrub in the ginger family that has been used in traditional Chinese medicine for thousands of years to relieve joint pain. It provides an excellent complement to ergothioneine because it works through a different mechanism to battle against free radicals and the inflammation they produce. Instead of lending electrons to stop the chain reaction, the turmeric in the present disclosure reduces the production of cytokines, the inflammation-inducing proteins that form when the body enters a period of oxidative stress. Cytokines recruit and direct white blood cells to the source of the stress, and that's what causes the swelling, redness, heat and pain.

The principal active compound in turmeric is curcumin. Clinical studies have shown that curcumin has extraordinary anti-inflammatory and antioxidant properties. Many of these studies suggest curcumin may be far more beneficial than first expected. While studying the effects of curcumin on intestinal inflammation, scientists at the University of South Carolina discovered that it not only reduces inflammation, but also reduced intestinal polyps (which can become cancerous) by an impressive 75% [5]. Doctors at Kirchberg Hospital in Luxembourg discovered that in addition to being safe and displaying “strong antioxidant properties,” curcumin has “huge potential” in the treatment of cancer [6]. Researchers at the University of Portugal found curcumin's cellular antioxidant defenses could be a useful approach toward anti-aging [7].

In addition to turmeric and ergothioneine, the present disclosure contains three additional natural botanical ingredients to help fight inflammation and the joint pain it can cause.

White willow bark comes from a tree native to Europe and Asia. The ancient Egyptians had been using it long before the Greek physician Hippocrates wrote about its medicinal value in the 5th century B.C. In 1829, European scientists identified a compound called salicin as the active ingredient in white willow bark. Bayer created a synthetic version of salicin-salicylic acid—and sold it as “aspirin.” Unlike this synthetic counterpart, natural white willow bark doesn't irritate the stomach because the salicin found in it is only converted to the acid form after the stomach absorbs it. The effectiveness of white willow bark is demonstrated by the double-blind study conducted in Germany by researchers at the Pharmazeutisches Institute. In that study, 78 arthritis sufferers were divided into two groups, with one group receiving 240 mg of white willow bark extract each day and the other group receiving a placebo. After 14 days, patients who received the white willow bark reported a 14% decrease in joint pain, as measured by the WOMAC Osteoarthritis Index (Western Ontario and McMaster Osteoarthritis Index). Those who received the placebo not only failed to experience pain relief, but actually reported that their pain had worsened [8].

The present formulation also includes two additional inflammation fighters. Cat's claw is a woody vine that grows wild in the rain forests and jungles of South America. Uncaria tomentosa, commonly known as cat's claw, is a medicinal plant native to Peru, which has been used for decades in the treatment of various inflammatory disorders. Uncaria tomentosa can be used as an antioxidant, has anti-apoptotic properties, and can enhance DNA repair, however it is best known for its anti-inflammatory properties. Treatment with Uncaria tomentosa extracts inhibits the production of the pro-inflammatory cytokine, TNF-alpha [9].

Chemical analysis of cat's claw shows that it contains novel plant chemicals called quinovic acid glycosides that have powerful anti-inflammatory properties. Studies have shown that cat's claw (and, especially, its glycosides) has the ability to reduce inflammation by as much as 89%. Because of its Peruvian origins, many of the cat's claw studies have been conducted in South America. One, at Universidad Nactional Mayor de San Marcos in Lima, Peru, involved 45 patients with osteoarthritis of the knee. Thirty of them were treated with cat's claw, while the other fifteen received a placebo. Researchers found that pain associated with activity was “significantly reduced” and that the benefits of cat's claw were experienced within the first week of therapy. They concluded, “Cat's claw is an effective treatment for osteoarthritis.” [10] Though it's been successfully used for many years, the benefits of cat's claw are still relatively unknown to most Americans.

Acai berry, the final inflammation fighter in the present disclosure, is the name given to a variety of similar fruits of palm trees native to South America. Acai is now widely recognized as having bona fide health benefits. A landmark study published in the Journal of Agricultural and Food Chemistry, for example, concluded that acai: 1) exhibited “exceptional activity against superoxide [a free radical] . . . the highest of any food reported to date”; 2) has antioxidants that are able to “enter human cells in a fully functional form and to perform an oxygen-quenching function at very low doses”; 3) was found to be “a potential cyclooxygenase (Cox)-1 and Cox-2 inhibitor.” [11] Another study, conducted in Brazil, determined that acai “decreased H2O2-induced damage of both lipids and proteins in all tissues tested.” [12] The berry helped protect tissues from damaging molecules. A leading Canadian research laboratory studied the antioxidant properties of acai using both in vitro and in vivo methodologies. The in vitro studies showed that the antioxidants in acai “penetrated and protected cells from oxidative damage” and cells “showed reduced formation of reactive oxygen species.” [13] In the in vivo studies (a randomized, double-blind, placebo-controlled, crossover trial) all participants displayed an increase in antioxidant levels in the blood. The active compounds in the acai berry are polyphenolic compounds like anthocyanin. This is the same group of flavonoids, although not necessarily the identical suite of compounds within that group, that account for the red to purple color in other fruits like grapes, blueberries, blackberries and raspberries.

The present disclosure also addresses other common causes of joint pain. Torn or thinning cartilage is another major cause of joint pain. Cartilage is the stiff connective tissue found between the bones at every joint. Even if the synovial membrane is 100% intact (a rarity among anyone over the age of 50), the joints are still subjected to a surprising amount of stress from ordinary day-to-day activities. Simply by walking, one can subject the hips and knees to stresses equal to roughly three times one's body weight. Cartilage can simply wear out as a result. The present disclosure includes glucosamine sulfate to provide the body with the building blocks it needs to naturally rebuild torn or thinning cartilage.

Glucosamine has been widely studied and long considered an important weapon in the war on joint pain. Even the Food and Drug Administration, which is openly skeptical about the benefits of dietary supplements, has been convinced to take a serious look at glucosamine. The FDA website states, “The scientific evidence . . . convincingly establishes that crystalline glucosamine sulfate, when given to individuals diagnosed with osteoarthritis, can prevent further joint degradation, can reverse the symptoms by minimizing the inflammation and restoring articular cartilage, can reduce joint pain and can result in increased joint function. Given the physiological mechanism of action of crystalline glucosamine sulfate and other factors, there also are sufficient data demonstrating the ability of crystalline glucosamine sulfate to be effective in reducing the risk of developing osteoarthritis.”

Another ingredient in the present formulation is collagen, the main component of ligaments, tendons, cartilage, bones, skin and blood vessels. Over time and as part of the normal aging process, collagen loses its elasticity. When this happens to skin, it wrinkles. When it happens to joint components, they become misaligned and the body experiences pain. Although the body can produce collagen if it has the right materials at the right time, it can rarely produce it fast enough to keep the ligaments and tendons as elastic as they need to be to operate at peak efficiency.

This occurs because the composition of a typical ligament consists of 90% type I collagen (mature tissue having the greatest tensile strength), 9% type II collagen (immature tissue having considerably less strength), and 1% fibroblast cells that produce collagen. With only 1% of the tissue mass devoted to collagen production, the ligament is hard pressed to produce enough of this vital protein.

Vitamin C is another ingredient in the present formulation. According to the National Institutes of Health, calcium ascorbate or vitamin C is “an essential nutrient in human diets, and necessary to maintain connective tissue and bone.” Multiple laboratory and clinical studies have shown promising results for the protective effect of vitamin C intake on the course of osteoarthritis, including disease incidence, progression and symptomatology [14].

The human body, unlike most animals, is unable to synthesize vitamin C. It utilizes dietary vitamin C in a number of ways. For example, vitamin C serves as an electron donor in the synthesis of collagen. It is also involved in the body's production of glycosaminoglycan, which is needed to produce and maintain the synovial fluid that lubricates joints. Although vitamin C is absolutely essential for healthy joints (and good health in general), the body is unable to produce it because of a genetic defect, so the body must acquire it through foods or supplements.

The Clearwater Osteoarthritis Study, a long-term study of 1,032 osteoarthritis sufferers conducted by researchers at the University of South Florida, demonstrates the crucial role vitamin C plays in joint health. Researchers found that people who take vitamin C supplements are 11% less likely to develop osteoarthritis in the knee [15]. Although the Clearwater Study focused on the knees, hundreds of other studies have demonstrated the importance of vitamin C to joint health in general—and to overall good health. Additionally, the study published in the Biochemical Journal shows that ergothioneine and vitamin C work together to fight free radical damage.

Vitamin B12 is another ingredient in the present formulation. The largest and most structurally complicated vitamin ever identified, B12 helps regulate the metabolism of every cell in the human body, as well as aiding in the synthesis of DNA. When vitamin B12 levels drop even slightly, you can experience a range of symptoms such as fatigue, depression and poor memory. A prolonged B12 deficiency can result in severe damage to the brain and nervous system [18]. In one case, an elderly man suffering from what doctors described as “cognitive and behavioral symptoms associated with neuropsychiatric disturbances.” After being treated with vitamin B12, his dementia disappeared and he was able to live a normal life. Seven years later he was still stable with no hint of dementia [19].

BioPerine® a proprietary black pepper extract (piperine), allows the body to absorb and fully utilize the other compounds in the present formulation. First, ergothioneine augments the impact of the other ingredients in the present disclosure, then BioPerine ensures the body is able to absorb the disclosed pain-relieving combination of ingredients. BioPerine is also the only form of black pepper to have undergone clinical studies in the U.S. to substantiate its safety and efficacy for nutritional use. One study measured the absorption of three distinct categories of nutrients with and without BioPerine. The categories evaluated were fat-soluble (betacarotene), water-soluble (vitamin B6) and a mineral (selenium). Gastrointestinal absorption of all the studied nutrients, as measured by amounts present in the blood, increased from 30% to 60% when administered with BioPerine as compared to the control group receiving the nutrient alone.

Origin and Biosynthesis of Ergothioneine

Ergothioneine has intrigued biochemists since its discovery in 1909 (1,2), in a fungal contaminant of rye grain, it was later found to be highly concentrated in the red blood cells of most animals and aggressively conserved by the body. Over the years, attempts have been made to characterize its origin, fate and function (2-4). Early attempts to identify ergothioneine as a vitamin were discontinued due to the lack of a well-defined animal model of ergothioneine deficiency, but some authors have suggested this role (5).

One role clearly elucidated for ergothioneine is that of a natural intracellular antioxidant (4,6) similar in many aspects to the major cytoplasmic thiol, glutathione (GSH). Several additional biochemical and biological activities have been attributed to ergothioneine but its physiological role beyond being an antioxidant must be clarified (7,8). It does appear to have a significant role in maintaining the function of erythrocytes as well as protecting them from oxidative damage (9, 10). The ability of ergothioneine to protect hemoproteins such as hemoglobin within erythrocytes (11, 12) against oxidation probably could explain the millimolar concentrations seen in these cells. The avidity by which dietary l-ergothioneine is incorporated into tissues, the tenacity with which it is retained and its unique non-uniform pattern of tissue distribution serve to support the physiological importance of this molecule.

Over the years, ergothioneine research has been limited by the lack of availability of a commercial source of pure synthetic material. Recently, researchers have successfully developed the first patented, commercially practical synthetic process for the industrial preparation of pure ergothioneine (13). The efficacy, clinical utility and commercial viability of other natural antioxidants in numerous disease states and in various OTC applications (cosmetics, preservatives, dietary supplements, etc.) are well known. Thus, this “recently rediscovered” natural antioxidant represents a new opportunity for development in areas where natural antioxidants have been commercially successful. In addition, because of its unique biological properties, ergothioneine holds potential for use in a wide array of oxidative stress-induced disorders.

Ergothioneine is synthesized by fungi and mycobacteria in soil, where it is readily absorbed by plants through their roots (2). In these micro-organisms it is biosynthesized from hercynine and cysteine (2,14, 82-83). Cysteine is the source of the sulfhydryl, and the introduction of sulfur is the last step in the pathway (83). To date, no mutant of these organisms has been identified which is lacking in ergothioneine biosynthesis.

No evidence exists for the direct biosynthesis of ergothioneine in animals or plants despite numerous studies attempting to identify a synthetic pathway (2, 15, 16, 17, 18). It seems likely that fungi are the source of most or all of the ergothioneine in plants and animals. It may be ingested in minute quantities either directly from foods such as mushrooms or plants and animals, or indirectly via assimilation by plants from the soil (2). Hence humans are auxotrophic for this compound and therefore must assimilate it through dietary intake of plants and/or animal foodstuffs.

Biological Distribution

Ergothioneine is a ubiquitous compound found in most animals and plants. The literature values for ergothioneine in various sources have been hindered by a lack of consistency in the methods used. While the molecule has been studied for almost a century, only recently have specific and sensitive methods been employed. There still remains much work to be done to paint an accurate picture of the levels of ergothioneine across a wide spectrum of source materials. Table I summarizes the distribution of ergothioneine in various tissues for 4 animal species studied from Melville done in the 1950s (2).

TABLE I
Concentrations of LE in various animal tissues.

| Tissue | Rat | Rabbit | Dog | Cat |
|---|---|---|---|---|
| Liver | 13.3 | 0.3 | 0.9 | 2.7 |
| RBC | 10.4 | 10 | 6.6 | 2.9 |
| Kidney | 4.3 | 0.3 | 1.6 | 3.1 |
| Heart | 1.5 | 2.7 | 8.9 | 0.0 |
| Lungs | 1.5 | 0.3 | 0.6 | 0.8 |
| Spleen | 1.1 | 1.0 | 1.1 | — |
| Testes | 0.0 | 0.1 | 0.0 | 0.0 |
| Muscle | 0.7 | — | — | — |
| Intestine | 0.6 | — | — | — |
| Stomach | 0.4 | — | — | — |
| Plasma | 0.0 | — | — | — |

Values expressed as mgs/100 gm fresh tissue.

Millimolar or submillimolar concentrations of ergothioneine have also been found in the bone marrow (19), cataract-free lens (20), and seminal fluid (21,22). While brain tissue is not reported in Melville's chart, Briggs (81) did an extensive study of ergothioneine distribution in ox brain and determined there were values that ranged from 0.36 to 0.03 mgs/100 gm depending upon the brain region examined. As seen, even on a relative basis, there is a tremendous range of distribution of ergothioneine across many species and the organ systems within the species. The existence of this data would support an evolved selective biological distribution of the molecule. The distribution pattern can be further evidence for a functional attribute. Ergothioneine is not known to be a significant energy source, or have a structural role; therefore the implication is that ergothioneine plays an important role in homeostasis as a regulator or effector. Its role as antioxidant is that of a maintenance function.

Ergothioneine is preferentially distributed to organ systems that are exposed to a high degree of oxidative stress. Blood concentrations of ergothioneine in almost every species investigated are in near millimolar range as shown in Table II (2). Levels in human blood cells were measured in healthy males from Saudi Arabia, and found to be 1-4 mg/100 mL. Levels in the blood appeared to peak during the ages of 11-18 years. Using TLC densitometry, levels found in rat tissues were especially high in the liver, and included: liver=7.82 mg/100 g, kidney=1.58 mg/100 g, testicle=0.5 mg/100 g, brain=0.41 mg/100 g, plasma=0.21 mg/100 g. An early study found ergothioneine in the brains of cats (3.0 μg/g), guinea-pigs (10 μg/g), as well as rats, mice, rabbits and sheep (the levels fall in between those of the cat and guinea-pig). Large amounts were found in the optic nerves of the rabbit (30 μg/g). It has some excitatory action on the electrical activity of the cerebellum in the decerebrate rabbit.

TABLE II
Concentration of l-ergothioneine in
the blood of various animals.
(mg/100 ml blood)

| Species | Ergothioneine |
|---|---|
| Man | 1-4 |
| Rat | 1-6 |
| Rabbit | 1-10 |
| G. Pig | 1-4 |
| Cat | 0.5-2 |
| Dog | 3-6 |
| Ox | 0.5-2 |
| Pig | 3-27 |
| Sheep | 2-6 |
| Fowl | 2-10 |

Biochemistry

Chemically, ergothioneine corresponds to the betaine of 2-thio-L-histidine. Although various synthetic compounds of this chemical class exist, ergothioneine is the only naturally occurring 2-thio-imidazole amino acid known to date. In aqueous solution, the tautomeric 2-thio-imidazole exists predominantly in the thione form. This explains why, unlike other alkylmercaptan antioxidants such as GSH, at physiologic pH ergothioneine does not auto-oxidize and is therefore very stable in aqueous solution.

The compound is extremely hydrophilic with a solubility limit of 0.9 M at room temperature (23). It is an excellent chelator of divalent metals especially copper and zinc (24, 25) and is remarkably stable to strong alkali, properties which further differentiate it from other biological thiols.

Over the years, several isolation techniques have been developed which employed starting materials such as blood, ergot or grains (2, 26, 27). Pig blood has the highest know amount of l-ergothioneine, but yields only 60 to 100 mg of 1-ergothioneine per liter of blood processed (2,26). Various fungal and mycobacterial sources are available for the production of ergothioneine, but the yields are too low to be commercially viable for an industrial production (28).

The synthesis of the natural 1-isomer of ergothioneine has proven to be quite difficult. Several attempts to synthesize ergothioneine (29, 30, 31) have failed to achieve satisfactory yields, were not industrially viable or resulted in partial (32) or complete racemization (33). OXIS International has developed the first efficient, commercially viable synthesis of ergothioneine, resulting in high yield (34) based on a new method of sulfur introduction into an imidazole ring (35).

Various methods have been employed to perform chemical analysis of ergothioneine. New and novel commercial assays for ergothioneine are currently in the validation stage. Early seed methods (58) were improved upon by a spectrophotometric assay developed by Carlsson (73) which eliminated cross reacting thiols and ascorbic acid with a Cu++ catalysed oxidation and the reaction of the non-labile LE with the reagent 2,2′ dipyridyl disulfide. HPLC methods have been the preferred means to accurately determine LE. Several methods have been published (59, 74, 75).

The role of ergothioneine as an antioxidant and cellular protectant have been well documented (2, 4). However, the unusual physico-chemical properties of ergothioneine and its preferential localizations within certain cells such as erythrocytes make it unique among naturally occurring antioxidants. The antioxidant properties of ergothioneine appear to be related to at least four activities, which include the molecule's ability to: 1) scavenge directly reactive oxygen species; 2) chelate various divalent metallic cations; 3) activate antioxidant enzymes such as glutathione peroxidase (Se-GPx) and MnSOD and to inhibit superoxide-generating enzymes such as NADPH-Cytochrome c reductase; 4) affect the oxidation of various hemoproteins such as hemoglobin and myoglobin.

In physiological concentrations, ergothioneine exhibits potent diffusion-controlled inactivation of hydroxyl radical (32, 84) and prevention of singlet oxygen production (38, 39). It does not function as a direct scavenger of superoxide anion, hydrogen peroxide or lipid peroxides. It also differs significantly from natural thiol-containing antioxidants in that it does not stimulate lipid peroxidation in the presence of ferric ions.

Ergothioneine is a powerful scavenger of hypochlorous acid (HOCI). Although many compounds can react with hypochlorous acid, few do so rapidly enough to be biologically meaningful. Alphal-protease inhibitor (API), the major inhibitor of serine proteases such as elastase, is an especially sensitive target of hypochlorous acid. Studies have shown that physiological concentrations of ergothioneine protect very efficiently API against HOCI-induced inactivation (6). However, since neutrophils are the principal source of hypochlorous acid in the body, one role for ergothioneine may be to protect circulating erythrocytes from the damage induced by neutrophils during normal function or pathologic inflammation.

Peroxynitrite, formed endogenously by the diffusion-limited reaction of nitric oxide with superoxide, is a potent oxidant which has been implicated in the pathophysiology of inflammation, ischemia-reperfusion injury, atherosclerosis, acute lung injury and sepsis. It has been shown that ergothioneine inhibits the peroxynitrite-mediated oxidation of amino acids such as the nitration of tyrosine and protects against the peroxynitrite-induced inactivation of alpha-1-antiproteinase (72).

Divalent metals such as iron, copper and zinc have been shown to participate in the production of destructive reactive oxygen species (ROS). Iron-induced conversion of hydrogen peroxide to the more damaging hydroxyl radical via the Fenton reaction is thought to be one of the primary mechanisms for initiation of free radical mediated tissue damage. In fact, iron is frequently used in many in vitro models as a means of generating free radicals to induce lipid peroxidation. In addition, copper is routinely employed as a catalyst for the evaluation of anti-lipidemic agents and their ability to inhibit LDL oxidation, which is believed to be the initiating event responsible for atherosclerotic plaque development. Compounds capable of complexing these metals (such as desferoxamine) have been shown to be highly effective in ameliorating various oxidative stress related diseases. However, their usefulness has been generally limited because of toxicological problems. In contrast, ergothioneine, a natural antioxidant found in human tissues, as a metal chelator (24,25), should have minimal toxicity.

Ergothioneine inhibits the NADPH-dependent enzymatic lipid peroxidation of hepatic microsomes after either NADPH or ascorbic acid challenge (39). Japanese researchers have found that ergothioneine markedly increased glutathione peroxidase and glutathione reductase activities in mouse liver cytosol fractions (39). Additionally mitochrondrial Mn-superoxide dismutase activity was nearly doubled by ergothioneine at a concentration of 12.5 mM but no increased activity of the cytosolic form of Cu/Zn SOD was noted. Thus, ergothioneine may also exert a significant biological benefit by stimulating cellular antioxidant systems against oxidative challenge. It remains to be determined if the enzymatic effects of ergothioneine are a consequence of direct enzyme stimulation and/or a consequence of the conservation if glutathione levels.

Another source of ROS in vivo is the exposure of hemoproteins such as hemoglobin or myoglobin to hydrogen peroxide (41, 42, 43). For example, oxyhemoglobin reacts with hydrogen peroxide to generate a high oxidation state iron species (FeIV=O) called ferryl-hemoglobin. This species plays a critical role in the lipid peroxidation of erythrocytes. Similarly myoglobin exposure to hydrogen peroxide causes oxidation of lipid peroxidation of erythrocytes. Similarly, myoglobin exposure to hydrogen peroxide causes oxidation of lipid membranes and contributes to the ischemia/reperfusion injury noted in ischemic heart or muscle (44). It has been shown that ergothioneine reduces the ferryl-myoglobin into metmyoglobin and by this way inhibits the lipid peroxidation (44) and protects against ischemia-reperfusion injury (45). Studies using the Langendorff model in rats have demonstrated an interaction of ferryl-myoglobin with ergothioneine in tissues, showing that ergothioneine (0.1 mM) protected the isolated rat heart against reperfusion injury as measured by LDH release. In a similar study on isolated rabbit heart, ergothioneine failed to protect against ischemiareperfusion injury (46). This discrepancy may be explained by several differences in the experimental models including species, perfusion methodology and duration of ischemia. However, researchers using LDH and CPK as measure of cardiac damage have confirmed the earlier isolated rat heart findings for ergothioneine.

Aruoma (71) tested ergothioneine for its ability to inhibit cell death caused by H2O2 and to inhibit DNA oxidation by peroxynitrite in a human neuronal hybridoma cell line in culture. Ergothioneine demonstrates secondary activities, which are independent of its antioxidant properties. Data suggest that it plays a role in the regulation of the energy requirements of erythrocytes (47). Since erythrocytes do not contain any mitochondria, they utilize the pentose-phosphate shunt to meet their energy requirements, resulting in the formation of lactic acid. When ergothioneine is added to hemolyzed erythrocytes or erythrocyte suspensions, increased lactate production is noted along with a concomitant decrease in intermediate glucose byproducts such as glucose-6-phosphate and fructose-6-phosphate. These biochemical changes are consistent with energy production by ergothioneine within erythrocytes. When ergothioneine was administered in the diet to rats it prevented a 40% starvation-induced diminution or erythrocytes lactate level that was noted in the control animals. In vitro incubation studies of human platelets demonstrated ergothioneine involvement in cellular energy production with increases in CO2 production from pyruvate noted concomitantly with a decreased production of lactate. These effects were similar to those noted for carnitine, suggesting that ergothioneine can also stimulate energy production in cells using normal cellular respiratory pathways (48). Therefore, ergothioneine may be required for these cells to maintain normal metabolic function, especially when exposed to high oxidative challenges. This may also explain why ergothioneine is selectively concentrated in the mitochondria. By utilizing the carnitine membrane shuttle, ergothioneine can enter from the outer to the inner membrane, possibly by a competitive binding mechanism.

Pharmacology

It has been shown that ergothioneine protected rats in vivo against hepatic injury associated with ethionine administration (a form of damage which occurs via formation of lipid peroxides). The hepatic injury produced after a single injection of ethionine was inhibited and hepatic lipid peroxide formation was reduced by pre-administration of LE (8 mg/100 gm body weight) for 7 days. A 40% increase in hepatic lipid peroxidation was noted in partially LE-deficient rats (<1 nmol/gm liver) as compared with dietary-augmented animals (1400 nmol/gm liver) (49).

The lens of the eye undergoes extensive oxidative challenge on a continuous basis. Cataract formation has been shown to result from a cumulative exposure of the lens to UV radiation. The resulting production of ROS over time depletes the antioxidant defenses normally present in the eye resulting in the gradual formation of cataracts. Ergothioneine has been found in high concentrations in the normal human eye (20) where it presumably functions as a protecting antioxidant. Table III summarizes the finding from a study (20), which examined the relationship between ergothioneine concentration and cataract development.

TABLE III
The concentration of LE in the lens of the human eye
as a function of cataract development

| Stage of Cataract | Ergothioneine | Number |
|---|---|---|
| Normal | 115.7 +/− 6.3 | N = 10 |
| Immature: | | |
| Nuclear | 94.2 +/− 7.3 | N = 20 |
| Cortical | 79.4 +/− 11.7 | N = 20 |
| Mature | 71.7 +/− 13.7 | N = 50 |
| Hypermature | 60.8 +/− 9.8 | N = 25 |

A clear reduction in ergothioneine concentration is evident over the course of cataract development. The greater decrease in ergothioneine noted in the cortical type of immature cataract is apparently related to the higher metabolic state associated with the cortex relative to the nucleus. The researcher also noted a progressive loss in ergothioneine concentrations as opacities increased.

Ergothioneine has also been studied in various radiation-induced damage models (50, 51, 84). UV-induced skin damage is known to be mediated via formation of various ROS, including singlet molecular oxygen, hydroxyl radical and superoxide. The effect of ergothioneine on inactivation of singlet oxygen was studied relative to other biological thiols (52). As compared to cysteine, N-acetyl-cysteine, glutathione and other synthetic mercaptans, ergothioneine was found to have the greatest affinity for singlet oxygen, i.e. 10 fold greater than glutathione. Although ergothioneine failed to protect mouse skin in vivo (53), it seems likely that it was not delivered through the stratum corneum. Ergothioneine was found to be a very potent radioprotector even at low concentrations in an in vitro study (85). Several patents have included ergothioneine as an active UV protectant.

Studies have shown that ergothioneine has a critical protective role in seminal fluid (21, 22). Ergothioneine is the predominant sulfhydryl in human, horse and pig semen. Its role is evidently to protect spermatozoa from oxidative stress, given the exceptionally high metabolic rate in sperm. Ergothioneine, as consequence of its antioxidant properties, counteracts the effects of hydrogen peroxide on spermatozoa viability and survival while also enhancing the viability of sperm during storage.

Animal studies have shown that as little as 1 part per 100,000 of ergothioneine in the diet can produce measurable changes in circulating whole blood levels (54). Following intravenous administration, pharmacokinetic studies (2) revealed a rapid disappearance of ergothioneine from the plasma into the organs, principally the liver. Neither hepatectomy nor nephrectomy altered this disappearance suggesting that tissue uptake rather than excretion occurred. These studies suggest that ergothioneine is rapidly absorbed and assimilated by various tissues. Complete depletion of ergothioneine from tissue stores has proven to be difficult (55, 56) even after extensive periods of starvation. The whole body half-life of ergothioneine in the rat is approximately 30 days (57). In rats, one study measured the fecal and urine loss of ergothionine, and calculated the absorptive gain per day on a controlled diet of ergthioneine supplementation. The gain was 20 μg/day for a 225 g rat, while the loss was 2 ug/per day, indicating a 90% accumulation rate. For humans, this would equal a retention rate of about six mg per day for 150 lb adult (54).

Ergothioneine uptake into red blood cells is believed to occur during erythropoiesis and it appears to remain present within the cell for its entire lifetime (2, 4, 10, 58). These data suggest that various processes exist within the erythrocyte for its conservation and/or regeneration. Over the normal 120-day life span of the erythrocyte, ergothioneine concentrations gradually decrease (9, 59) probably as a consequence of cumulative oxidative exposure. This gradual decline in ergothioneine concentrations within the erythrocyte, despite normal dietary intake, plus the high ergothioneine concentrations found in bone marrow, add credence to the theory that ergothioneine enters erythrocytes during hematopoiesis. Numerous studies have found that only minute amounts (<1%) of ergothioneine can directly penetrate the erythrocyte membrane (2, 10, 58, 59, 60). Although the mechanism of uptake has not been elucidated, the fact that erythrocytes from most species contain high concentrations of ergothioneine suggests that one of its principal biological activities is associated with erythrocyte function and regulation. Ergothioneine penetration into seminal fluid and most other tissues besides erythrocytes is rapid.

No documented negative side effects or know toxic pathways have been reported as a result of ergothioneine administration. Kunisaki speculated that intake of nitrites in the diet may enhance the formation of nitrosamines (86). No physiological nitrosamine product was ever isolated or levels measured to support the theory.

Metabolism

The metabolic fate of ergothioneine in humans has never been studied. Animal studies have been performed mainly in rat and rabbit models. Ergothioneine given in the diet at low concentrations is very efficiently absorbed and retained in the rat. However, many studies have been performed using bolus injections intraperitonealy or directly into the blood. These conditions most certainly will not yield the same metabolic fate in humans, as the low dose dietary route of entry.

Unfortunately, there is a lack of uniformity in procedures used to study the fate of radiolabled ergothioneine. However, the results of several researchers provide some insight into the transformations that are possible. The metabolism of ¹⁴C-dLE was examined in the rat (61). Apparently the most important step in ergothioneine metabolism is the loss of the thiol group. The liver was identified as the principal site of ergothioneine metabolism where it is converted to the non-thiol containing derivative hercynine, which is subsequently excreted. This conclusion is supported by studies with ³⁵S-ergothioneine (15) where 35-65% of the sulfur label was recovered in the urine as free sulfate. Urinary excretion is the principal route of bolus injected ergothioneine elimination, with 60% of the dose being recovered in approximately 6 hours. About one-third of the urine products were identified as ergothioneine and another one-third as hercynine.

Microbial metabolism may occur to some extent in the gut. However, efforts to measure synthesis, even at very high levels of radioactivity, were unable to measure any significant incorporation of radiolabled histidine into isolated ergothioneine. Because humans do have the genetic machinery to synthesize ergothioneine, the species has not evolved a specialized degradation pathway. Instead, what has evolved are systems to sequester and maintain exogenously derived ergothioneine. It is not clear exactly what transport systems exist. The mitochondrial carnitine shuttle may be one of those systems.

The metabolism and biochemistry of ergothioneine within the context of its function as an antioxidant is still being explored. Many groups have looked at ergothioneine and its interactions with free radicals. Asmus (66) found that ergothioneine interacts effectively with oxidizing radicals (hydroxyl, azide, and carbontetrachlorideperoxide), and in the presence of ascorbate is regenerated back. Ascorbate performs a similar function in converting tocopherol quinone back to Vitamin E. In an animal experimental model of diabetes, Aruoma and coworkers have shown that antioxidants (BHT, Vitamin E, Vitamin C) decrease the rate of embryo malformations (67). The same group was also able to show that ergothioneine can also act to reverse the developmental defects to almost that of the control group (68).

Toxicology

Extensive animal toxicity studies with ergothioneine have not been reported. However, results from numerous experiments have produced no deleterious effects following ergothioneine ingestion in either humans or animals. Ergothioneine is present in various foodstuffs (2) and is readily absorbed after ingestion as part of a normal diet. The risk of any serious toxicity associated with the compound is probably minimal especially given its high circulating and tissue levels. Abbreviated pharmacological studies (62, 63) originally conducted in the 1920's and 1930's failed to show any deleterious effects of ergothioneine at physiologically relevant concentrations. The carcinogenic potential of the compound is apparently low with several investigators reporting that ergothioneine actually protects against mutagen production (64, 65).

Health Benefits and Disease Prevention

Aruoma's group has shown that ergothioneine inhibits both H₂O₂ and Tumor necrosis factor-alpha-mediated imposed oxidative stress in an IL-8-chloramphenicaol acetyl transferase (CAT) reporter system in A549 cells. This anti-inflammatory response was found to be due to a lowering of transcription NF-kB and Activator Protein-1, resulting in abolishing the transcription activation of the pro-inflammatory cytokine Interleukin-8 (69).

Aruoma has also shown a neuroprotective effect of ergothioneine in the rat retina in an in vivo N-methyl-D-aspartate excitotoxicity system (70). Cell counts revealed that 81% of ganglion cells and 43% of nonganglion cells were lost as a result of the treatment. In rats treated with ergothioneine, the losses were dropped to 44% and 31% respectively. Aruoma also measured Ameloid Precursor Protein (APP), and found significant decreases. The protein has been associated with Alzheimer's Disease. A similar protective effect was found against the oxidative base modifications induced by peroxynitrite on calf-thymus DNA, and nitration of tyrosine and inactivation of alpha-1-antiproteinase (72).

A study of 115 subjects with various cataractous lense was compared with 10 normal lenses (76). The quantity of ergothioneine (expressed as mg LE/100 gm) was markedly reduced in all four types of cataractous lenses (immature nuclear 94.2, Cortical 79.6, Matue 71.7, hypermature 60.8) compared to controls (115.7). The reduction may reflect an inability to deal with oxidative stress since GSH levels are lowered as well (76). Aruoma (77) has data to show that when rats are injected intraperitoneally with ergothioneine, it can be incorporated into the retina and reverse the effects of NMDA toxicity. These findings indicate that ergothioneine may play some protective role in the formation, and potential reversal of cataracts.

Diabetes Melitus has been associated with oxidative stress (88-89). Use of antioxidants may have a profound positive influence in controlling the oxidative stress problems associated with diabetes. An early study showed that some diabetic patients had elevated levels of ergothioneine (78). This led Epand to speculate that ergothioneine could be inducing diabetes through chelation of zinc (79). Later experiments performed by Epand found there to be no statistical significance between blood levels of ergothioneine between diabetic and non-diabetics (80).

Dosage Levels

It is difficult to determine how much ergothioneine is being taken in by the average consumer today. This will undoubtedly vary widely from culture and region. If we use only the United States, and make some conservative assumptions about the types and quantities of foods that are consumed, we can develop some range estimates based on food composition and retention/absorption data in the rat. While it is highly unlikely that ergothioneine is consumed at even a nominal level in the typical diet, for the purpose of estimation if the average adult consumes 1 pound (approximately 0.5 Kg) of LE containing foodstuff per day, and the average amount is the average of cereal (oat value of 17 mg/kg) and meat (10 mg/Kg), we arrive at an amount of [(17+10)/2] mg/kg×0.5 Kg, or approximately 7 mg per day. For a 150 lb individual, this would calculate to 104 ug ergothioneine/kg-day body weight. The rat absorption study of Mayumi et al. (59) concluded that a 225 gm rat absorbed 90% of the ergothioneine provided in the diet, which was 20 ug/day, or 89 ug ergothioneine/kg-day. Thus, the values are very similar. If the human absorption capacity is similar to that found in the rat, then the vast majority of ingested ergothioneine is retained. Therefore, supplementation on the order of 5-10 mg per day would certainly be within an order of magnitude, and more likely a factor of 2-3 or more of what the average human adult is consuming in their diet already.

Mayumi concluded that it would take the rat approximately 2 months to double the total body amount of ergothioneine at 89 ug ergothioneine/kg-day supplementation rate. If the same is true in humans, then this slow rate of accumulation would allow a very comfortable adjustment period for determining any potential side effects due to the supplementation. A two-month adaptation period is quite long compared to most pharmaceuticals. For example, antidepressants will usually ramp from half to full dosage up over a period of 30 days.

Melville points out that because ergothioneine is not synthesized by plants, but is actively transported from the soil through the roots, there may very well exist a wide variation in the amount of ergothioneine in selected vegetable sources, depending upon the soil conditions of the region and the growing conditions associated with various plant sources. Consumption of vegetables in the United States is chronically below recommended levels in all 50 states. The variation in daily intake will also affect the levels of ergothioneine found in meat sources as well.

Specialized Unique Antioxidant

Ergothioneine is a highly protective, nontoxic, naturally occurring antioxidant that is not easily auto-oxidizable in aqueous solutions. It is readily water soluble, reaches near millimolar concentrations in selected tissues, and stimulates the natural antioxidant defenses within cells. The benefits of natural antioxidants such as vitamin C and vitamin E in cancer, aging and general health are well known. Newer natural antioxidants such as pyrogallol, lipoic acid and ubiquinone are now being introduced into the market. Ergothioneine is unique among antioxidants in that it chelates heavy metal, while protecting cells (principally erythrocytes) from ROS damage.

Dermal Protectant/Anti-Aging

The role of free radicals in ultraviolet light induced skin damage, and the role of UVB radiation in skin cancer is well known. Ergothioneine, because of its ability to minimize the formation of various ROS and to protect cells from radiation induced damage, is currently being evaluated in novel liposomal delivery systems. Since ergothioneine is a natural product, the goal of this program is to develop OTC sunscreens and/or protective cosmetics.

Ophthalmic Benefit

The observation that the eye contains extremely high concentrations of ergothioneine that decrease during cataract development suggest that ergothioneine plays a critical role in the protection of the eye. The effect of UV radiation on the eye and its association with cataracts is well known. The aim is to develop an ophthalmic product to replenish the loss of ergothioneine noted during cataract development. The aqueous solubility of ergothioneine and its stability offer major advantages since the ideal ophthalmic should be available to topical administration.

Energy Enhancer

Evidence from several sources points to a positive effect of ergothionine in increasing the availability of cellular energy sources. Kawano (47) speculated that ergothioneine might stimulate Phosphofructokinase activity or increase glucose absorption in erythrocytes as a way to explain the significant increase in glycolytic activity measured after addition of 1 mM ergothioneine to intact human red cell cells in vitro. A similar report was made by Chiba (87), but the compound was described only as a synthetic sulfhydryl compound. As was discussed in the Biochemistry section, there is good evidence for increased respiration in platelets, with ergothioneine playing a role similar to carnitine in shuttling acyl-CoA derivatives across the mitochondrial membrane (48).

Organ Preservation

The availability of viable organs for transplantation is currently limiting the number of organ transplants that can be conducted in the US. Preservation of the available tissues and prolongation of their viability is an important determinant in both the ultimate success of the procedure and the number of patients that can receive transplants. Specifically, liver viability is limited to 8 hours, which severely limits the transport of these organs. Although, current preservation solutions are formulated to include antioxidants such as glutathione, the instability of these compounds (significant degradation begins to occur immediately after manufacture) limits their usefulness in protecting organs from oxidative damage. Glutathione even in refrigerated preservation solutions is readily oxidized, to its disulfide form. The later form is cytotoxic and also facilitates inflammation-induced proteolysis. Ergothioneine, a stable water-soluble thiol-containing antioxidant that also chelates metal ions, could be an ideal to replace glutathione in this mixture.

Table IV lists the preferred embodiment of a capsule supplement containing ergothioneine.

TABLE IV
Capsule Formulation

| INGREDIENTS | WEIGHT | |
|---|---|---|
| Vitamin B-12 (Cyanocobalamin) | 250 | mcg |
| Glucosamine Sulfate (from Glucosamine 2 HCl) | 500 | mg |
| Calcium Ascorbate (from Ascorbic Acid and Calcium | 50 | mg |
| Ascorbate) | | |
| Turmeric Extract (95% curcuminoids) (Curcuma | 50 | mg |
| longa) (root) | | |
| Bioperin ® Black Pepper Extract (95% Piperine) (Piper | 2.5 | mg |
| nigrum) (fruit) | | |
| Hyal-Join TM (60% Hyaluronic Acid, 30% Collagen, | 2.5 | mg |
| 10% Glycosaminoglycans) | | |
| ERGO (L-Ergothioneine) | 250 | mcg |
| Proprietary Blend | 150 | mg |
| Cat's Claw Extract 4:1 | | |
| Acai Berry (10%, 3%, 1%) | | |
| White Willow Bark Extract (10% Salicin) (Salix alba) | | |

Other ingredients may include gelatin, microcrystalline cellulose, maltodextrin, dicalcium phosphate dihydrate, silicon dioxide, magnesium stearate, and guar gum.

The formulation above was the result of a study undertaken to evaluate the anti-inflammatory properties of the unique thiol, L-ergothioneine, in concert with other ingredients with anti-inflammatory compounds, as well as compounds providing structural building blocks for maintenance and repair of cartilage. The following protocol will describe an open-label clinical pilot study to explore the effects of the disclosed supplement's consumption on chronic, painful joint conditions that affect range of motion, activities of daily living, and inflammation.

Disclosed herein is a new formulation for, among other things, joint health and reducing inflammation. It also contains an interesting blend of other ingredients (glucosamine, chondroitin, collagen, and hyaluronic acid) and ingredients with strong antioxidant and anti-inflammatory capacity. This study protocol will be the first study to examine the effects of the disclosed formulation on chronic joint pain in a systematic manner, and link to blood tests involving antioxidant status and inflammatory markers.

Twelve human subjects were tested over a period of six weeks. Recruiting of volunteers happened via NIS Labs. Subjects were be monitored at baseline, and after one, three, and six weeks. A blood sample was taken at each visit. The inclusion of a one-week assessment allowed evaluation of how rapidly a reduction of chronic pain was observed. The inclusion of weekly phone interviews allowed collection of some pain/activities of daily living data via questionnaires. Simplified brief questionnaires were used at the phone interviews.

After the period of consumption was completed, and each study participant exited the study, there was a follow-up six weeks later (“12 wk”), where a full exam and questionnaires again assessed pain and activities of daily living. This follow-up time point helped evaluate how chronic pain may return after consumption has ended. The consent form was worded to reflect the usual wording that “a study participant may withdraw from the study at any time”, but with the addition that if participants withdraw before the 12 weeks are completed, researchers would like the opportunity to perform the full exit evaluation at that time point.

Twelve healthy subjects of both genders were selected if they were between the ages of 30-65 and had mild-moderate complaints of chronic pain affecting range of motion in specific anatomical areas. Volunteers were not selected if they regularly consumed, for the past two months, supplements containing glucosamine, chondroitin, hyaluronic acid; if they regularly consumed supplements/juices with a high antioxidant content; if they were undergoing stressful life events that would compromise compliance; if they had ongoing intensive medical treatment for other diseases (cancer, viral disease); if they were pregnant, lactating, or trying to become pregnant; or if they had allergies to ingredients in the test product.

Particularly in a study where the outcome measures are focused on inflammation and oxidation, various stressors needed careful monitoring. This included exercise, mental stress, diet, supplement intake, and sleep. At each visit, the study coordinator asked questions regarding current health, meals, stimulants, stress level, and sleep.

At each NIS Labs visit (i.e. study start (Day 0), and at two, and four weeks), the study coordinator assisted the participants in filling in the pain questionnaires, and conducted an interview to allow other health changes to be monitored systematically.

The following questionnaires and tracking were performed at each visit: 1) NIS Labs daily intake questionnaire (includes tracking of use of recent illness, medications, recent events, perceived health changes); 2) WOMAC; 3) pain scores (using visual analogue scales) for areas identified as primary and secondary pain complaints; 4) questionnaire on activities of daily living; 5) NIS Labs questionnaire of minor health complaints (digestion, skin problems, mood, sleep, etc.); and 6) blood pressure.

Serum was collected and banked as part of the study. Later serum testing may include: high sensitivity C-reactive protein; serum antioxidant protection status; testing of ergothioneine levels; serum lipid peroxidation status; and serum cytokine profile.

It should be appreciated that the compositions containing ergothioneine and one or more compounds selected from the group consisting of vitamin B-12, glucosamine sulfate, calcium ascorbate, turmeric extract, black pepper extract, hyaluronic acid, cat's claw extract, acai berry; and white willow bark extract act synergistically to provide an unexpected benefit for a variety of health related factors, including but not limited to brain health, joint health, eye health, mitochorial optimization/improvement and reduction of inflammation and pain in a mammal.

Range of Motion

The evaluation of ROM was conducted in a very detailed manner, where not only a person's major area of discomfort was evaluated, but the entire vertical axis of the body was studied, from the neck to the knees. The rationale behind this detailed assessment is that often a person's primary complaint will lead to a compensated posture and ROM of other anatomical areas as the person strives to put less pressure on the painful area. This complete assessment (30 distinct ROM measurements) optimizes the ability to show significant changes as a result of product consumption.

The J-Tech Tracker Freedom dual digital inclinometry assessment of ROM was analyzed in two different ways: i) Overall ROM for each anatomical area, and ii) Specific assessment of each person's problem areas.

The anatomical areas are: cervical (neck); shoulders; thoracic (upper body/torso); upper extremities (shoulders); lumbar (lower back); and lower body (hips, knees). Most people may have more severe pain and ROM problems in the lumbar and lower parts of the body than the upper parts of the body. Areas that did not have problems at study start should not be expected to improve, whereas areas that had limited ROM at study start were carefully monitored for improvements.

A second analysis focused on the specific types of motion that were impaired for each person, such as bending the head backwards, rotating the shoulder, or extending the right knee. Each volunteer was analyzed for percent improvement from his or her own baseline (study start). These individual percent changes in ROM for a single discreet anatomical area were then averaged for all study participants.

Baseline ROM: A range of motion exam was performed at study entry for several purposes: to make the subject used to the examination procedure so subsequent readings are more accurate and not affected by learning; to evaluate subject-specific pain and ROM complaints, based on which some personal protocol variations may be incorporated into the testing; to gather information on past and current health issues, and to verify that no health issues would compromise their participation in this study.

Subsequent ROM visits: The subsequent visits for ROM evaluation involved data collection on ROM using the established protocol for each person. Dr. David Ager, DC, at Cascade Spine and Rehabilitation clinic, using the J-Tech dual digital inclinometry, performed all ROM assessments.

The ROM measures are listed here: Cervical Flexion; Cervical Extension; Cervical Lateral Left; Cervical Lateral Right; Cervical Rotation Left; Cervical Rotation Right; Left Shoulder Abduction; Left Shoulder Adduction; Left Shoulder Internal Rotation; Left Shoulder External Rotation; Right Shoulder Abduction; Right Shoulder Adduction; Right Shoulder Internal Rotation; Right Shoulder External Rotation; Thoracic Flexion; Thoracic Rotation Left; Thoracic Rotation Right; Lumbar Flexion; Lumbar Extension; Lumbar Lateral Left; Lumbar Lateral Right; Left Hip Internal Rotation; Left Hip External Rotation; Left Knee Flexion; Right Hip Internal Rotation; Right Hip External Rotation; Right Knee Flexion.

The components, steps, features, objects, benefits and advantages that have been discussed are merely illustrative. None of them, nor the discussions relating to them, are intended to limit the scope of protection in any way. Numerous other embodiments are also contemplated. These include embodiments that have fewer, additional, and/or different components, steps, features, objects, benefits and advantages. These also include embodiments in which the components and/or steps are arranged and/or ordered differently.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The phrase “means for” when used in a claim is intended to and should be interpreted to embrace the corresponding structures and materials that have been described and their equivalents. Similarly, the phrase “step for” when used in a claim embraces the corresponding acts that have been described and their equivalents. The absence of these phrases means that the claim is not intended to and should not be interpreted to be limited to any of the corresponding structures, materials, or acts or to their equivalents.

Nothing that has been stated or illustrated is intended or should be interpreted to cause a dedication of any component, step, feature, object, benefit, advantage, or equivalent to the public, regardless of whether it is recited in the claims.

### Other Publications

(1) TANRET C., Sur une base nouvelle retiree du seigle ergote, L-Ergothioneine Compt. Rend. Acad. Sci., 149:222-224, 1909.
(2) MELVILLE D. B. L-Ergothioneine. Vitam & Horm, 17:155-204, 1958.
(3) KONISHI T. et al. Studies on L-Ergothioneine II, Origin of L-Ergothioneine in animal organs. Vitamins, 46:127-130, 1972.
(4) HARTMAN P. E. L-Ergothioneine as antioxidant. Meth. Enzy., 186:310-318, 1990.
(5) REGLINSKY J. et al., Spin-echo 1H-NMR detected response of L-Ergothioneine to oxidative stress in the intact human erythrocyte. Mag. Res. Med. 6:217-223, 1988.
(6) AKANMU D. et al., The antioxidant action of L-Ergothioneine. Arch. Biochem. Biophys., 288:10-16, 1991.
(7) BRUMMEL M. C., In search of a physiological function for L-Ergothioneine. Med. Hypoth, 18:351-370, 1985.
(8) BRUMMEL M. C., In search of a physiological function for L-Ergothioneine. Med. Hypoth., 30:39-48, 1989.
(9) TOUSTER O. J., The L-Ergothioneine content of human erythrocytes, the effect of age, race, malignancy and pregnancy. J. Biol. Chem., 188:371, 1951.
(10) KUCHEL P. W., CHAPMAN B. E., Proton NMR studies of creatine in human erythrocytes., Biomed. Biochem. Acta, 42:1143-1149, 1983.
(11) ARDUINI A. et al., Possible mechanism of inhibition of nitrie-induced oxidation of oxyhemoglobin by L-Ergothioneine and uric acid., Arch Biochm. Biophys., 294:398-402, 1992.
(12) SPICER S. S., WOOLEY J. G., KESSLER V., L-Ergothioneine depletion in rabbit erythrocytes and its effects of methaemoglobin formation and reversion. Proc. Soc. Exp. Biol. & Med., 77:418-420,1951.
(13) YADAN J. C. and XU J., U.S. Pat. No. 5,438,151, 1995.
(14) ISHIKAWA Y., et al., Participation of an intermediate sulfoxide in the enzymatic thiolation of the imidazole ring of hercynine to form L-Ergothioneine., J. Biol. Chem., 249:4420-4427, 1974.

### (15) HEATH, Biochem J., 54:689-697, 1953.

### (16) HEATH, et al., Biochem J., 54:606-611, 1953.

(17) MELLVILLE D. B., HORNER W. R. and LUBSCHEZ R. J., Biol. Chem, 206:221-228, 1954.
(18) MELLVILLE D. B., et al. , Biol. Chem., 213:61-68, 1955.
(19) LUTWAK-MANN C., Some aspects of phosphorus metabolism in bone marrow. Changes in the content of phosphate compounds and reducing substances in bone marrow and spleen caused by ionizing radiation and other factors, which depress the function of blood forming tissues. Biochem. J., 52:356-364, 1952.
(20) SHUKLA Y., KULSHRESTHA O. P., and KHUTETA K. P., L-Ergothioneine content in normal and senile human cataractous lenses. Ind. J. Med., Res., 73:472-473, 1981.
(21) MANN T. and LEONE E., Studies on the metabolism of seman 8, L-Ergothioneine as a normal constituent of boar seminal plasma. Biochem. J., 53:140-148, 1953.
(22) HAAG F. M. and Mc LEOD J., Relationship between nonprotein sulfhydryl concentration of seminal fluid and motility of spermatozoa in man. J. Appl. Physiol., 14:27-30, 1959.

### (23) The Merck Index, 10th Edition, po.529, Merck & Co, Rahway, N.J., 1983.

(24) HANLON D. P., Interaction of L-Ergothioneine with metal ions and metalloenzymes. J. Me. Chem., 14:1084-1087, 1971.
(25) MOTOHASHI N. et al., Metal complexes of L-Ergothioneine. Chem. Pharm. Bull., 22:654-657, 1974.
(26) BENEDICT S. R., NEWTON E. B. and BEHRE J. A., J. Biol. Chem., 67:267-277,1926.
(27) BALDRIDGE R. C., Blood L-Ergothioneine and dietary oats. Fed. Proc., 13:178, 1954.
(28) MIYOSHI T. and SAKAI H., L-Ergothioneine by microbial fermentation. JP patent, number 450623, 1968.
(29) ASHLEY J. N. HARINGTON C. R., J. Chem. Soc.,: 2586, 1930.

### (30) HARINGTON C. R. and OVERHOFF J., Biochem J., 27:338, 1933.

(31) JACKSON A. O., J. Biol. Chem., 103:191, 1933.
(32) HEATH H., LARSON A. and REMINGTON C., J. Chem. Soc.,: 2215-2217, 1951.
(33) SUNKO D. E. and WOLF G., J. Chem. Soc.,: 4405-4406, 1958.
(34) XU J. and YADAN J. C., Synthesis of l-(+)-ergothioneine. J. Org. Chem., 60,20:6296-6301, 1995.
(35) XU J. and YADAN J. C., A new and convenient synthesis of imidazole-2-thiones from Imidazole. Synlett, 3:239-241, 1995.
(36) MOTOHASHI N. and MORI I., Thiol-induced hydroxyl radical formation and scavenger effect of thiocarbamides on hydroxyl radicals. J. of Inorg. Biochem., 26, 205-212, 1986.
(37) HAN J. S., Effects of various chemical compounds on spontaneous and hydrogen peroxideinduced reversion in strain TA 104 of Salmonella typhimurium Mutation Res., 266, 77-84, 1992.
(38) HARTMAN P. E. et al., Scavenging of singlet molecular oxygen by imidazole compounds: High and sustained activities of carboxy terminal histidine dipeptides and exceptional activity of imidazole-4-acetic acid Photochem. And Photobiol., 51, 59-66,1990.
(39) DAHL T. A. et al., Some prevalent biomolecules as defenses against singlet oxygen damage Photeochem. And Photobiol, 47,357-362, 1988.
(40) KAWANO H. et al., Studies on LE XI, Inhibitory effect on lipid peroxide formation in mouse liver. Chem. Pharm. Bull., 31:1682-1687, 1983.
(41) CLEMENS M. R. et al., Biochem. Pharm., 34:1339-1341, 1985.
(42) GALARIS D. et al., Arch Biochem. Biophys., 262:221-231, 1988.

### (43) PUPPO A. and HALLIWELL B., Biochem J., 249: 185-190, 1988

(44) ROMARO R. et al., The reactivity of thiols and disulfides with different redox states to myoglobin. J. Biol. Chem., 267:1680-1688, 1992.
(45) ARDUINI A., EDDY L. and HOCHSTEIN P., The reduction of ferryl-myoglobin by LErgothioneine, a novel function for LE. Arch. Biochem. Biophys., 281:41-43, 1990.
(46) CARGNONI A. et al., In vitro administration of L-Ergothioneine failed to protect isolated ischaemic and reperfused rabbit heart Biochimica and biophysica Acta, 1270, 173-178, 1995.
(47) KAWANO H. et al., Studies on LE, some effects of L-Ergothioneine on glycolytic metabolism in RBS of rats. Chem. Pharm. Bull., 30:2611-2613, 1982.
(48) KERBY G. P and TAYLOR S. M Effect of carnitine and L-Ergothioneine on human platelets metabolism. Pro. Soc. Exp. Biol. Med., 132:435-439, 1969.
(49) KAWANO H. et al., Studies of LE X, Effect of L-Ergothioneine on the hepatic drug metabolizing enzyme system and on experimental hepatic injury in rats. Chem. Pharm. Bull., 31: 1676-1681, 1983.
(50) MOTOHASHI N. et al., Radioprotectent effect of L-Ergothioneine on gamma-irradiation of metmyoglobin. Chem. Pharm. Bull, 25: 2516-2523, 1977.
(51) HARTMAN P. E. HARTMAN Z. and CITARDI M. J., L-Ergothioneine, histidine and two naturally occurring histidine dipeptides and radioprotectors against gamma radiation inactivation of bacteriophages T4 & P22 Rad Res., 114:319-330, 1988.
(52) ROUGEE M. et al., Deactivation of singlet molecular oxygen by thiols and related compounds, possible protectors against skin photosensitivity. Photochem. Photobiol, 47:485-489, 1988.
(53) VAN DEN BROCHE L. T. et al., UV-radiation protecting efficacy of thiols, studies with UVA induced binding of 8-MOP and CPZ in rats epidermal biomacromolecules in vivo. Int. J. Rad. Biol., 63:493-500, 1993.
(54) POTTER V. R. and FRANKE K. W., The effect of diet upon blood thioneine (L-Ergothioneine) of the albino rat. J. Nitrit, 9:1, 1935.
(55) BALDRIDGE R. C. & LEWIS H. B., Diet and L-Ergothioneine content of blood. J. Biol. Chem., 202: 169, 1953.
(56) MELVILL D. B. et al., Fed. Proc., 14:254, 1955.
(57) KAWANO H. et al., Studies on L-Ergothioneine VI, Distribution and fluctuations of L-Ergothioneine in rats. Chem. Pharm. Bull., 30:1760-1765, 1982.
(58) JOCELYN P. C., The distribution of L-Ergothioneine in blood as determined by new method of estimation. Biochem. J., 70:656-660, 1958.
(59) MAYUMI T. et al., Studies on L-Ergothioneine V, Determination by HPLC & application to metabolic research. Chem Pharm. Bull., 26:3772-3778, 1978.
(60) MELVILLE D. B., OTHEN C. C. and KOVALENKO V., J. Biol. Chem., 216:325-331, 1955.
(61) WOLF G., BERGAN G., and SUNKO D. E., metabolism studies with 14-C l-ergothioneine. Biochem Biophys. Acta, 54:287-293, 1961.
(62) TAINTER M. L., Notes on the pharmacology of L-Ergothioneine. Proc. Coc. Exp. Biol. Me., 24:621, 1926.
(63) TARBUCCHI E. boll-Soc. Ital. Biol. Sper., 11:117, 1936.
(64) MAYUMI T. et al., Studies on LE VIII, Preventive effects of L-Ergothioneine on cadmium induced teratogenesis. Chem. Pharm. Bull., 30:2141-2146, 1982.
(65) HARTMAN Z. AND HARTMAN P. E. Interception of some directing acting mutagens by L-Ergothioneine. Envir. Mol. Mutagen., 10:3-15, 1987.
(66) ASMUS K D. Et al., One-electron oxidation of ergothioneine and analogues investigated by pulse radiolysis: redox reaction involving ergothioneine and vitamin C. Biochem. J. 15:315, 1996.
(67) VIANA M. et al., Oxidative Damage in Pregnant Diabetic Rats and their Embryos. Free Radical Biol. Med. 11:1115-21, 2000.
(68) GUIJARRO M V. Et al., Effects of Ergothioneine on Diabetic Embryopathy. Submitted to Food and Chemical Toxicology, 2001.
(69) GILMOUR P S. Et al., Ergothioneine inhibits Oxidative stress and TNF-A-induced NF-kB/AP-1 Binding and Interleukin-8 Release in Alveolar Epithelial Cells. Submitted for Publication
(70) MONCASTER J A. Et al., Ergothioneine protects neurons against N-methyl-D-aspartate excitotoxicity in an in vivo retinal model. Submitted for publication to Neuroscience Letters, 2002.
(71) ARUOMA O I, Spencer J P E, Mahmood N. Protection Against Oxidaitve Damage and Cell Death by the Natural Antioxidant Ergothioneine. Food and Chem. Tech. 37:1043-1053, 1999.
(72) ARUOMA O I, Whiteman M, England T G, Halliwell B. Antioxidant Action of Ergothioneine assessment of its ability to scavenge peroxynitrite. BBRC 231:389-391, 1997.
(73) CARLSSON J, KIBRSATN, M P J, BROCKLEHURST K. A Convenient Spectrophotometric Assay for the Determination of L-Ergothioneine in Blood. Biochem. J. 139:237-242, 1974.
(74) NEWTON G L et al. Low Molecular weight Thiols in Streptomycetes and their Potential Role as Antioxidants. J. Bacteriol. 175:2734-2742, 1993.
(75) KANEKO I. Et al. Quantitative Determination of Ergothioneine in Plasma and Tissues by TLCDensitometry. Chem. Pharm. Bull. 28:3093-3097, 1980.
(76) XIE P Y. Et al. Glutathione and Glutathione-Related enzymes in Human Cataractous Lenses. Opthalmic Res. 23:133-140, 1991.
(77) ARUOMA O I. et al. The retina as a novel in vivo experiemental model for studying the neuroprotective effects of antioxidants: ergothioneine treatment protects neurons in CNS against N-methyl-Aspartate (NMDA) excitotoxicity. Submitted to Free Radical Biology and Medicine, 2002.
(78) FRASER R. Blood Ergothioneine levels in Diabetes Meellitus. J. Lab Clin. Med. 35:960, 1950.
(79) EPAND, R M. The Role of Dietary Ergothioneine in the Development of Diabetes Mellitus. Medical Hypotheses 9: 207-213, 1982.
(80) EPAND R M, EPAND R F. Study of the Ergothioneine Concentration in the Blood of Individuals with Diabetes Mellitus. J. Clin. Chem. Biochem. 26:623-626, 1988.
(81) BRIGGS I. Ergothioneine in the Central Nervous system. J. Neurochem. 19:27-35, 1972.
(82) DUPRE S, SPOTO G, SOLINAS S P. Sulfur Amino Acids: Biochemical and Clinical Aspects, pp343-353, 1983.
(83) GENGHOF D S, VAN DAMME O. Biosynthesis of ergothionine from endogenous Hercynine in Mycobacterium Smegmatis. J. Bacteriol. 95:340-344, 1968.
(84) HAN J-S. Effects of various chemical compounds on spontaneous and hydrogen peroxide-induced reversion in strain TA104 of Salmonella Typimurium. Mutation Research 22: 77-84, 1992.
(85) HARTMAN P E, HARTMAN Z, CITARDI M J. Ergothioneine, Histidine, and Two naturally occurring dipeptides as radioprotectors against gamma-irradiation inactivation of Bacteriophages T4 and P22. Radiation Res. 114:319-330, 1988.
(86) KUNISAKI N, HAYASHI M. Effects of ergothioneine, Cysteine, and Glutathione on Nitrosation of Secondary amines under Physiologic Conditions. JNCI 65: 791-794, 1980.
(87) CHIBA T. Chem. Pharm. Bull. 22:439, 1974.
(88) BAYNES, J W. Reactive Oxygen in the aetiology and complications of diabetes. In Drugs, Diet and Disease, Vol. 2: Permagon Press, London, pp203-240, 1995.
(89) BAYNES J W, THORPE S R. Role of Oxidative Stress in Diabetes. Diabetes 48:1-9, 1999.
[1] Petrella, R. J. “Hyaluronic acid for the treatment of knee osteoarthritis: long-term outcomes from a naturalistic primary care experience.” American Journal of Physical Medicine and Rehabilitation. 2005 Apr. 84(4):278-83.
[2] Aruoma, O. I. “Antioxidant action of ergothioneine: Assessment of its ability to scavenge peroxynitrate.” Biochemical and Biophysical Research Communications. 1997. 231:389-391.
[3] Deiana, M. et al. “L-ergothioneine modulates oxidative damage in the kidney and liver of rats in vivo: studies upon the profile of polyunsaturated fatty acids.” Clinical Nutrition. 2004.23(2):183-93.
[4] Asmus, K. D., et al. “One-electron oxidation of ergothioneine and analogues investigated by pulse radiolysis: redox reaction involving ergothioneine and vitamin C.” Biochemical Journal. 1996. 315(Pt 2):625-9.

### [5] Murphy, E. A., Davis, J. M., McClellan, J. L., Gordon, B. T., Carmichael, M. D. “Curcumin's Effect on Intestinal Inflammation and Tumorigenesis in the Apc(Min/+) Mouse.” J Interferon Cytokine Res. 2010 Oct 15.

[6] Kelkel, M., Jacob, C., Dicato, M., Diederich, M. “Potential of the dietary antioxidants resveratrol and curcumin in prevention and treatment of hematologic malignancies.” Molecules. 2010 Oct. 12. 15(10):7035-74.
[7] Lima, C. F., Pereira-Wilson, C., Rattan, S. I. “Curcumin induces heme oxygenase-1 in normal human skin fibroblasts through redox signaling: Relevance for anti-aging intervention.” Mol Nutr Food Res, 2010 Oct. 11.
[8] Schmid, B., Ludtke, R., Selbmann, H. K., et al. “Efficacy and tolerability of a standardized willow bark extract in patients with osteoarthritis: randomized placebo-controlled, double blind clinical trial.” Phytotherapy Research, 2001. Jun. 15(4):344-50.
[9] Allen-Hall, L., Cano, P., Amason, J. T., Rojas, R., Lock, O., Lafrenie, R. M. “Treatment of THP-1 cells with Uncaria tomentosa extracts differentially regulates the expression if IL-Ibeta and TNF-alpha.” J Ethnopharmacol. 2007 Jan. 19. 109(2):312-7.
[10] Piscoya, J., Rodriguez, Z., Bustamante, S. A., Okuhama, N. N., Miller, M. J., Sandoval, M. “Efficacy and safety of freeze-dried cat's claw in osteoarthritis of the knee: mechanisms of action of the species Uncaria guianensis.” Inflamm Res. 2001 Sep. 50(9):442-8.
[11] Schauss, A. G., Wu, X., Prior, R. L., Ou, B., Huang, D., Owens, J., Agarwal, A., Jensen, G. S., Hart, A. N., Shanbrom, E. “Antioxidant capacity and other bioactivities of freeze-dried Amazonian palm berry, Euterpe oleraceae mart. (acai).” J Agric Food Chem, 2006 Nov. 1. 54(22):8604-10.
[12] Spada, P. D., Dani, C., Bortolini, G. V., Funchal, C., Henriques, J. A., Salvador, M. “Frozen fruit pulp of Euterpe oleraceae Mart. (Acai.) prevents hydrogen peroxide-induced damage in the cerebral cortex, cerebellum, and hippocampus of rats.” J Med Food. 2009 Oct. 12(5):1084-8.
[13] Jensen, G. S., Wu, X., Patterson, K. M., Barnes, J., Carter, S. G., Scherwitz, L., Beaman, R., Endres, J. R., Schauss, A. G. “In vitro and in vivo antioxidant and anti-inflammatory capacities of an antioxidant—rich fruit and berry juice blend. Results of a pilot and randomized, double-blinded, placebo-controlled, crossover study.” J Agric Food Chem, 2008 Sep. 24. 56(18):8326-33.
[11.4] Peregoy, J., et al. “The effects of vitamin C supplementation on incident and progressive knee osteoarthritis: a longitudinal study.” Public Health Nutrition. 2010 Aug 16:1-7.

### [11.5] Id.

[16] McBride, H. M., Neuspiel, M., Wasiak, S. “Mitochondria: more than just a powerhouse”. Curr. Biol. 2006. 16 (14): R551.
[17] Aruoma. O. I., Spencer, J. P., Mahood, N. “Protection against oxidative damage and cell death by the natural antioxidant ergothioineine.” Food and Chemical Toxicology. 1999. 37(11): 1.043-53.
[18] See, e.g., Herbert V., Vitamin B12 in Present Knowledge in Nutrition, 17th ed. Washington, DC: International Life Sciences Institute Press. 1996.
[19] Blundo C. Marin D, Ricci M. “Vitamin B12 deficiency associated with symptoms of frontotemporal dementia.” Neurol Sci. 2010 Oct. 7.

We claim:

1. A composition for reducing or treating inflammation and pain in a mammal, the composition comprising an effective amount of L-Ergothioneine and an effective amount of two or more compounds selected from the group consisting of:
vitamin B-12;
glucosamine sulfate;
calcium ascorbate;
turmeric extract;
black pepper extract;
hyaluronic acid;
collagen;
glycosaminoglycans;
cat's claw extract;
acai berry; and
white willow bark extract.

2. The composition of claim 1, wherein the effective amount of L-Ergothioneine is 50 mcg to 1000 mcg daily.

3. The composition of claim 1, wherein the effective amount of vitamin B-12 is 50 mcg to 5000 mcg, the effective amount of glucosamine sulfate is 50 mg to 5000 mg, the effective amount of calcium ascorbate is 5 mg to 500 mg, the effective amount of turmeric extract is 5 mg to 500 mg, the effective amount of black pepper extract, hyaluronic acid, collagen and glycosaminoglycans is 0.25 mg to 25 mg each and the effective amount of cat's claw extract, acai berry and white willow bark extract is 10 mg to 1500 mg each daily.

4. The composition of claim 1, wherein the composition further includes a pharmaceutically acceptable carrier.

5. The composition of claim 1, wherein the composition further includes vitamin C.

6. The composition of claim 1, further comprising one or more compounds selected from the group consisting of:
gelatin;
microcrystalline cellulose;
maltodextrin;
dicalcium phosphate dihydrate;
silicon dioxide;
magnesium stearate; and
guar gum.

7. A composition comprising an effective amount of Ergothioneine and an effective amount of two or more compounds selected from the group consisting of:
cat's claw extract;
acai berry; and
white willow bark extract.

8. The composition of claim 7, wherein the effective amount of Ergothioneine is 50 mcg to 1000 mcg daily.

9. The composition of claim 7, wherein the effective amount of cat's claw extract, acai berry and white willow bark extract is 10 mg to 1500 mg each daily.

10. The composition of claim 7, wherein the composition further includes a pharmaceutically acceptable carrier.

11. The composition of claim 7, wherein the composition further includes vitamin C.

12. The composition of claim 7, further comprising one or more compounds selected from the group consisting of:
gelatin;
microcrystalline cellulose;
maltodextrin;
dicalcium phosphate dihydrate;
silicon dioxide;
magnesium stearate; and
guar gum.

13. A method of reducing inflammation and pain in a mammal, the method comprising:
a. administering to a mammal an effective amount of a composition containing L-Ergothioneine and one or more compounds selected from the group consisting of vitamin B-12, glucosamine sulfate, calcium ascorbate, turmeric extract, black pepper extract, hyaluronic acid, collagen, glycosaminoglycans, cat's claw extract, acai berry; and white willow bark extract.

14. The method of claim 13, wherein the effective amount of L-Ergothioneine is 50 mcg to 1000 mcg daily.

15. The method of claim 13, wherein the effective amount of vitamin B-12 is 50 mcg to 5000 mcg, the effective amount of glucosamine sulfate is 50 mg to 5000 mg, the effective amount of calcium ascorbate is 5 mg to 500 mg, the effective amount of turmeric extract is 5 mg to 500 mg, the effective amount of black pepper extract, hyaluronic acid, collagen and glycosaminoglycans is 0.25 mg to 25 mg each and the effective amount of cat's claw extract, acai berry and white willow bark extract is 10 mg to 1500 mg each daily.

16. The method of claim 13, wherein the composition further includes a pharmaceutically acceptable carrier.

17. The method of claim 13, wherein the composition further includes vitamin C.

18. The method of claim 13, further comprising one or more compounds selected from the group consisting of:
gelatin;
microcrystalline cellulose;
maltodextrin;
dicalcium phosphate dihydrate;
silicon dioxide;
magnesium stearate; and
guar gum.
